(19) United States
(12) Patent Application Publication
(10) Pub. No.: US 2013/0344005 A1
(43) Pub. Date: Dec. 26, 2013

(54) BOLUS FOR TREATING HYPOCALCAEMIA IN RUMINANTS

(71) Applicant: LABORATOIRES ADITEC, Dinard (FR)

(72) Inventor: Gilles LE JEAN, Saint Coulomb (FR)

(21) Appl. No.: 13/923,569

(22) Filed: Jun. 21, 2013

(30) Foreign Application Priority Data
Jun. 22, 2012 (FR) 1255941

(51) Int. Cl. A61K 33/10; A61K 31/194

(52) U.S. Cl. CPC A61K 33/10; A61K 31/194
USPC 424/44; 424/687

(57) ABSTRACT

The subject of the invention is a veterinary product or a nutrition product intended in particular for the prevention and treatment of hypocalcaemia in ruminant animals. The product is in the form of a bolus comprising calcium carbonate and calcium formate. Advantageously, two of these boluses are concomitantly administered, one being a rapid-release effervescent bolus and the other being a slow-release bolus.

The subject of the invention is a veterinary product or a nutrition product intended in particular for the prevention or treatment of hypocalcaemia in ruminant animals.

In high producing cows, the beginning of lactation poses a major problem in terms of calcium metabolism. The rapid increase in lactation as soon as calving takes place rapidly increases calcium requirements. The cow, which is coming out of gestation, is not used to releasing its body calcium stores, and a few days are necessary for the mechanisms of this storage release to be set up. A critical period of a few days therefore ensues, just after calving, during which the cow which has just calved struggles to maintain its blood calcium at a satisfactory level, i.e. about 80 mg/l. When the blood calcium level drops below 80 mg/l, the cow enters into subclinical hypocalcaemia, which has few symptoms but which is known to impair milk production performance levels. When the blood calcium level drops below 50 mg/l, the cow is no longer steady on its feet and lies down. If nothing is done, survival prognosis becomes an issue. The cow is then at the clinical stage of vitular fever also known as “milk fever” or “postpartum hypocalcaemia”.

Clinical vitular fever is generally treated by intravenous injection of calcium in order to restore the blood calcium level. This curative procedure falls within the competence of veterinary medicine.

In order to limit the frequency of these extreme and serious cases, it is possible to perform nutritional prevention of the risk of vitular fever by treating the cows around calving before they develop subclinical vitular fever.

The first nutritional supplements used in this indication were gels of mineral or organic calcium salts. By way of example, the Calform® product from Bayer Animal Health is an oral gel sold in bottles of 350 ml and containing 165 g of calcium formate, i.e. 50 g of calcium, calcium formate being recognized as a form that is less irritant than calcium chloride and that has good bioavailability. The dosage is set up as follows: 350 ml the day before calving, 350 ml at calving, 350 ml 12 hours after calving and 350 ml 24 hours after calving. This technique is effective but has two drawbacks. The product is administered with slow swallowing and the animal must have its head raised, which requires the animal to be restrained during the swallowing time. In reality, the farmer does not always adhere to these dispensing recommendations and false swallowing ensues, i.e. the product passes into the trachea with serious and painful pulmonary consequences for the animal.

In order to overcome these drawbacks, products have been developed in bolus form, i.e. in the form of large oral tablets. These boluses have the advantage of being much quicker to administer (about one minute) and without the risk of false swallowing. A product of this type is sold under the brand name Bovikalc® by Boehringer Ingelheim.

Bovikalc® weighs 190 g and contains 42 g of calcium in the form of calcium chloride (67% of the formula) and of calcium sulphate (27% of the formula). The dosage follows a rhythm close to that of the gels, i.e. at least one bolus before calving and one bolus 12 hours later.

The Bovikalc® galenical form unquestionably represents progress compared with the Calform® product, but the composition of the product is not satisfactory. Bovikalk® provides calcium in two forms:
calcium chloride which is admittedly a source of rapidly metabolized calcium, but which is aggressive to the mucosae and in particular the oesophageal mucosa. For this reason, Bovikalc is covered with a thin protective shell and the information sheet stipulates that said shell must remain intact until administration to the animal;
calcium sulphate, well known as a constituent of plaster, and which acts more as a plasticizer for the bolus than a nutritional substance.

Finally, effervescent boluses have already been proposed, but they are not administered dry. On the contrary, they are diluted in the drinking water of the animals.

The digestive system of ruminant animals has the particularity of comprising an enormous reservoir called the reticulorumen, stratified into three phases of different consistency: a gas phase above a solid phase which itself floats above a liquid phase. Only the liquid phase, loaded with microparticles, passes into the rest of the digestive tract.

A solid food or active ingredient resides in the reticulorumen for a longer period of time than a liquid and its absorption into the blood system is consequently slower. Conversely, an active ingredient dispensed in liquid form, or in water-dispersible form, comes into contact with the animal's blood system faster, either by means of absorption by the ruminal wall, or by means of a faster passage for the purposes of absorption in the subsequent parts of the digestive tract.

As regards the forced administration of active ingredients to ruminant animals, and for reasons of practicality, the use of the “bolus” forms is advantageous. The bolus is a solid tablet placed by the operator beyond the fold of the tongue of the animal, which the latter swallows, placing it by itself in the reticulorumen. A bolus is not in the form of a gel.

Given the particularities of the reticulorumen described above, the bolus form does not allow active ingredient absorption as quickly as the liquid forms. However, it is easier to make a ruminant animal ingest a bolus than to make it ingest a liquid. As it happens, in some diseases or in some disorders, in particular vitular fever, the rapidity of absorption of the active ingredient and the consistency of the blood concentration thereof over time are determining factors for guaranteeing the efficacy of the treatment.

Given the prior art, there is therefore still a need to take advantage of the ease of administration of the bolus while providing calcium with nonaggressive and bioavailable salts. Moreover, it is important to sequence the calcium intakes in order to avoid a sawtooth blood calcium level, without however multiplying the administration actions by the carer.

It has been discovered in the context of the present invention that a bolus comprising the combination of calcium carbonate and calcium formate makes it possible to increase the bioavailability of the calcium and to accelerate its absorption compared with the existing bolus products for the same indication.

For the purposes of the invention, a bolus is a tablet of powders which is intended to be administered to an animal by oral route, in dry state, that is to say without having been previously dissolved in water. This tablet is substantially free of water and dissolves in the rumen fluid of the rumen of the animal after administration.

It was also discovered that it is possible to avoid calcium sawtooth by coadministration of two bolus forms solids composition of different calcium salts, which had never been previously proposed.

Finally, it has been found that a bolus comprising calcium formate, a basic calcium source and an organic acid different from formic acid capable of reacting with the basic calcium source in the presence of water effervesced in the rumen of the animal after administration, so that calcium absorption rate is higher.

According to the first aspect, the invention relates to a first bolus and a second bolus, which each comprise at least one calcium salt, the first bolus releasing calcium into the body of a ruminant animal to greater than the second speed bolus. This set of bolus can be used as a dietary supplement in a method of ruminant nutrition. The bolus set may also be used for the treatment or prevention of hypocalcaemia in a ruminant animal, the two boluses being administered concomitantly in dry form to said ruminant.

According to a second aspect, the invention relates to a bolus, preferably in the form of a solid tablet that is substantially free of water, including calcium formate, a basic source of calcium and a mineral or organic acid different from formic acid capable of reacting with the basic calcium source in the presence of water. The bolus has the advantage of generating a calcium salt in the rumen, after its administration in the mouth of the animal. The calcium salt may be formed during a reaction of effervescence which increases its absorption rate in animals. The bolus can be used as a dietary supplement in a method of nutrition of ruminants. It can also be used for treatment or prevention of hypocalcaemia in a ruminant animal, being administered orally in dry form.

Finally, in a third aspect, the invention relates to a bolus, preferably in the form of a solid tablet substantially free of water, including calcium formate and calcium carbonate.

In the following description, the term “bolus” can be used to designate the first or second bolus of the first aspect of the invention, the bolus of the second aspect of the invention or the bolus of the third aspect of the invention.

The bolus advantageously has a density and a weight such that it falls directly into the liquid phase of the reticulum.

The bolus is preferably anhydrous or substantially free of water, meaning that it contains less than 5% by weight of water, preferably less than 1% by weight of water. It is preferably in the form of a solid tablet that can be administered orally to the ruminant.

The calcium salts contained in the bolus are preferably selected from organic and inorganic calcium salts, preferably from organic calcium salts, in particular salts of gluconate, formate, citrate, carbonate and calcium stearate.

The calcium carbonate preferably represents from 5% to 50% by weight of the weight of the bolus. The calcium formate preferably represents from 5% to 60% by weight of the weight of the bolus. According to one embodiment, the calcium carbonate represents from 35% to 40% by weight of the weight of the bolus, and the calcium formate represents from 5% to 15% by weight, preferably from 7% to 12% by weight, of the weight of the bolus. Such amounts can be used to prepare a bolus with high calcium release rate.

According to another embodiment, the calcium carbonate represents from 5% to 15% by weight of the weight of the bolus, and the calcium formate represents from 50% to 55% by weight of the weight of the bolus. Such amounts can be used to prepare a bolus with a low calcium release rate.

The bolus, when it is prepared by compression of powders, advantageously comprises any additive required for forming it, in particular fillers and lubricants required for the compression of the powders so as to obtain a solid form which can be administered in the form of a large tablet. The fillers are generally chosen from sugars, such as lactose or sorbitol. The lubricants are, for example, calcium stearate or magnesium stearate.

The lubricant is advantageously a salt of fatty acid and calcium, such as calcium stearate.

The bolus is advantageously not coated in a protective film at the time of its administration; its weight is generally between 15 to 200 g.

The bolus preferably contains between 10 g and 50 g of calcium, more preferably between 15 and 25 g of calcium, in the form of calcium carbonate and of calcium formate. The weight of the calcium carbonate and of the calcium formate therefore preferably represents between 40% and 70% by weight of the weight of the bolus.

The first and/or the second bolus may be free of calcium propionate and free of calcium oxide.

A subject of the invention is also the bolus which has just been described in the second and third aspect of the invention, for use thereof in the treatment or prevention of hypocalcaemia in ruminant animals.

For the purposes of the invention, the hypocalcaemia is not necessarily a metabolic disorder. Its prevention does not systematically come under the prevention of a disease. The hypocalcaemia for the purposes of the invention is a blood calcium level less than or equal to 80 mg/l.

The ruminant animals are, for example, cattle, members of the sheep family, members of the goat family or buffalo, more preferably females exhibiting increased calcium requirements, in particular females undergoing lactation, for example dairy cows.

The bolus of the invention is of most particular interest in treatment for preventing hypocalcaemia in female cattle before calving or at the beginning of lactation, and in particular for preventing vitular fever.

For the purposes of the invention, vitular fever is a metabolic disorder which occurs around the time dairy cows drop their young.

The bolus is preferably administered just before calving, as soon as the signs indicating calving occur or during calving by the cow. This provision restores the blood calcium level and maintains it above the critical threshold, preferably above 80 mg/l, thus reducing the risk of vitular fever. The bolus is preferably administered before any clinical sign of hypocalcaemia or before any drop in blood calcium level, in order to avoid any subsequent additional treatment, and in particular calcium gluconate infusions.

In one embodiment, a second bolus is administered at least 12 hours after the first.

Thus, a subject of the invention is also a method for the nutrition of ruminant animals comprising the administration of a bolus as described previously.

According to this aspect of the invention, the bolus can promote maintenance of a good general condition in order to support milk production or to increase milk production.

According to one embodiment, the bolus also comprises an inorganic or organic acid so that the bolus becomes effervescent once it is in contact with the reticulo-ruminal fluid after its administration.

Use is preferably made of an organic acid, and more preferably anhydrous citric acid, since citric acid reacts with calcium carbonate to form calcium citrate, which is more absorbable in the intestine than calcium carbonate.

It is preferred for the amounts of organic acid and of calcium carbonate to be such that the effervescence reaction occurs for at least 10 minutes, preferably at least 15 minutes and more preferably at least 20 minutes in the rumen of the animal.

In this embodiment, the rapid release of calcium in the form of a formate salt is combined with the release of a highly absorbable calcium salt, the effect of which is the rapid and significant increase in blood calcium level.

The effervescence releases soluble calcium formate which is hydrolysed to Ca²⁺ absorbed either by the ruminal wall or in the intestine. The effervescence converts the calcium carbonate to calcium citrate, which is not very soluble but more absorbable in the intestine than the carbonate.

The effervescent bolus makes it possible to reduce the risk of milk fever, by virtue of a rapid and high absorption of calcium in formate and citrate form.

According to one embodiment, the bolus is not effervescent.

In one advantageous embodiment, a subject of the invention is a set of a first bolus and a second bolus, which each comprise at least one calcium salt, the first bolus releasing calcium in the body of a ruminant animal at a rate greater than that of the second bolus. The two boluses advantageously have different calcium release kinetics, so that the first, for example, releases calcium rapidly and the second releases calcium after a delay in time, so as to prolong the effect of the first, and to maintain a steady blood calcium level.

The two boluses have different compositions, preferably while being consistent with the bolus that has been described previously. The first and second boluses are preferably anhydrous, in the meaning that they contain less than 1% by weight of water. They can contain two or three different calcium salts. Calcium salts can be inorganic or organic and are preferably selected from organic calcium salts, in particular, salts such as gluconate, formate, citrate, carbonate and calcium stearate. The first or second boluses, or both, may contain calcium formate, calcium carbonate and calcium citrate.

Both bolus are preferably consistent with bolus has been described previously.

According to a particular embodiment, at least one of the two boluses contains calcium carbonate and/or calcium formate. For example, each comprise two bolus calcium formate and calcium carbonate, the first bolus comprising calcium carbonate as major component by weight, and the second bolus comprising calcium formate as the major compound by weight. The first bolus is preferably anhydrous, in the sense that it contains less than 1% by weight of water. The calcium salt is preferably chosen from organic and inorganic salts, in particular calcium gluconate, formate, citrate, carbonate and stearate salts.

The two boluses are preferably in accordance with the bolus which was described previously.

According to one embodiment, the first bolus can, for example, be an effervescent bolus and the second bolus preferably only becomes effervescent when it reaches the rumen, since it is intended for a slower calcium release.

The first bolus preferably contains calcium formate and an effervescent couple (preferably inorganic or organic acid, for example anhydrous citric acid and a basic calcium source, for example calcium carbonate or calcium oxide (CaO)). The first bolus falls into the system where it begins the process of effervescence in contact with the juice reticulo-rumen. The acid, for example citric acid, is combined with the basic calcium source, for example calcium carbonate to form calcium salt that can ne absorbed by the digestive system of the animal, for example calcium citrate, known for its bioavailability. At the same time, the effervescence phenomenon accelerates diffusion of calcium formate in the reticulo-rumen juice and thus its access to absorption sites.

The first bolus can alternatively contain, calcium citrate, calcium formate, a third calcium salt and optionally a pair of product which effervesces in contact with water.

The first bolus dissolves in vitro at 40° C. in rumen juice in a period of between 5 minutes and 1 hour, for example between 10 and 40 minutes.

The second bolus preferably contains calcium formate and calcium carbonate, but it is preferably not effervescent. The second bolus that liberates calcium at a lower rate than the first bolus, once administered to the animal, drops into the reticulum where it begins the process of disintegration by simple contact with the reticulo-ruminal juice. The second bolus dissolves in vitro at 40° C. in rumen juice in a period of between 1 and 10 hours, for example between 6 and 8 hours.

According to one embodiment of the invention:
in the first bolus, the calcium carbonate represents from 35% to 40% by weight of the bolus, and the calcium formate represents from 5% to 15% by weight of the weight of the bolus, and
in the second bolus, the calcium carbonate represents from 5% to 15% by weight of the bolus, and the calcium formate represents from 50% to 55% by weight of the weight of the bolus.

The combination of the two boluses can provide, in a single administration, calcium carbonate (which is partially converted to calcium citrate) and calcium formate, all with different release rates, thus making it possible to spread out the calcium absorption between two intakes, and to avoid variations in blood calcium level in the animal that are too abrupt.

The administrations of the first bolus and of the second bolus are advantageously carried out simultaneously using an applicator which makes it possible to administer the two boluses in a single action; failing this, they are successive and not more than 15 minutes apart, preferably not more than 5 minutes apart.

At least two sets of boluses as described previously are preferably administered. A first set of boluses is preferably administered to the female ruminant before calving, as soon as the first signs of calving appear, just after calving or at the beginning of lactation. A second set of boluses is advantageously administered at least 12 hours after the first.

The first bolus can be administered in dry form (without water) and dissolves in the rumen with effervescence.

This provision of calcium, in two boluses at different disintegration rate, and three different calcium salts, is a systematic innovation on the market.

This innovation is effective as a nutrition product, in particular for preventing vitular fever in dairy cows.

A subject of the invention is also a method for administering a nutrition product to ruminant animals, comprising the concomitant dry administration of two boluses each comprising at least one calcium salt, the first bolus releasing calcium in the body of the ruminant at a rate greater than that of the second bolus.

According to the second aspect of the invention, the bolus is in the form of a solid tablet, substantially free of water, comprising calcium formate, a basic calcium source and a organic or inorganic acid different from formic acid capable of reacting with the basic calcium source in the presence of water.

In this bolus, the basic calcium source is preferably calcium carbonate, and preferably is from 5 to 50% by weight of the bolus. Calcium formate can represent 5 to 60% by weight of the bolus.

The organic acid is capable of reacting with the basic calcium source in the presence of water to form a calcium salt that is absorbed by rumen wall or intestine of a ruminant animal. The acid is preferably citric acid, and reacts with the basic calcium source to generate calcium citrate in the presence of water, particularly in the rumen juice.

In this embodiment, the bolus preferably contains calcium stearate as a lubricant.

According to the third aspect, the bolus is in the form of a solid tablet substantially free of water comprising calcium formate and calcium carbonate. The features that have been described above in connection with the bolus apply to this third aspect. In particular, the calcium carbonate may represent from 5 to 50% by weight of the weight of the bolus, and calcium formate can represent 5 to 60% by weight of the weight of the bolus. Finally, the bolus can be used in the treatment or prevention of hypocalcaemia in ruminant animals.

The features which have been previously described in relation to the boluses apply to the administration method of the invention.

The invention is illustrated in greater detail by the following examples.

### EXAMPLE 1

### Rapid-Release Calcium Bolus According to the Invention

A 105 g effervescent bolus having the following composition was prepared. The percentages are by weight.

| | |
|---|---|
| calcium carbonate | 37.7% |
| citric acid | 15.0% |
| sorbitol | 36% |
| calcium formate | 10% |
| calcium stearate | 1.3% |

The in vitro tests in rumen juice at 40° C. showed complete dissolution of the bolus in 30 minutes.

### EXAMPLE 2

### Slow-Release Calcium Bolus According to the Invention

A 105 g effervescent bolus having the following composition was prepared. The percentages are by weight.

| | |
|---|---|
| calcium carbonate | 10% |
| sorbitol | 33.9% |
| calcium formate | 52.7% |
| PEG 6000 | 2.4% |
| calcium stearate | 1% |

The in vitro tests in rumen juice at approximately 40° C. showed complete dissolution of the bolus in 7 hours.

### EXAMPLE 3

### Animal Study

An experiment was carried out by a veterinary field doctor, in a normal farming situation, the objective being to measure the change in blood calcium level over time in four cows and to evaluate the degree of prevention of vitular fever.

The cows chosen all exhibited risks or a history of vitular fever. Boluses of Examples 1 and 2 were concomitantly administered, one or more times, and their blood calcium level was measured over time.

Cow 1: Race PrimHolstein, 5 years old, in third lactation, coming
from a heavy lactation (12932 kg) and too fatty at calving

| | Simultaneous | | |
|---|---|---|---|
| | administration | Blood | |
| | of the two | calcium | Verbatim observation |
| | boluses | level (mg/l) | by the vet |
| Before calving | Yes | Nd | Situation at risk. |
| Calving | Yes | 80 | Good results. The |
| Calving + 3 h | | 82 | calcium level is |
| Calving + 6 h | | 80 | holding. No |
| Calving + 12 h | Yes | 77 | subsequent |
| Calving + 24 h | | 72 | problems. |

Cow 2: Race PrimHolstein, 4 years old, in third lactation,
coming from a heavy lactation (9265 kg).

| | Simultaneous | | |
|---|---|---|---|
| | administration | Blood | |
| | of the two | calcium | Verbatim observation |
| | boluses | level (mg/l) | by the vet |
| Calving − 2 h | Yes | 81 | Situation at risk. |
| Calving | Yes | 77 | Good results. |
| Calving + 3 h | | 82 | The blood calcium level |
| Calving + 5 h | | 95 | is holding. The |
| Calving + 11 h | Yes | 84 | treatment is effective. |
| Calving + 24 h | | 75 | No subsequent problem |

Cow 3: Race PrimHolstein, 4 years old, in third lactation,
coming from a normal lactation (7903 kg).

| | Simultaneous | | |
|---|---|---|---|
| | administration | Blood | |
| | of the two | calcium | Verbatim observation |
| | boluses | level (mg/l) | by the vet |
| Calving − 3 h | Yes | 84 | Situation at moderate |
| Calving | Yes | 86 | risk. Good results. The |
| Calving + 3 h | | 85 | blood calcium level is |
| Calving + 5 h | | 84 | holding. The treatment is |
| Calving + 11 h | Yes | 86 | effective. No subsequent |
| Calving + 24 h | | 81 | problem |

Cow 4: Race PrimHolstein, 6 years old, in fourth lactation,
coming from a normal lactation (8555 kg).

| | Simultaneous | | |
|---|---|---|---|
| | administration | Blood | |
| | of the two | calcium | Verbatim observation |
| | boluses | level (mg/l) | by the vet |
| Before Calving | No | Nd | Situation at medium risk. |
| Calving | Yes | 78 | The treatment is |
| Calving + 3 h | | Nd | effective. No subsequent |
| Calving + 6 h | | 81 | problem |
| Calving + 12 h | Yes | 75 | |
| Calving + 26 h | | 77 | |

In the four cases above, the blood calcium level remained virtually linear at around 80 mg/litre. The results demonstrate the zootechnic effectiveness of the bolus of the invention with respect to the prevention of vitular fever. This effectiveness is reinforced when the administration of the bolus is carried out before the hypocalcaemia is too established.

1. Method of administering a bolus to a ruminant animal, comprising a step of administering a first bolus to the ruminant via oral route,
wherein the first bolus is in the form of an anhydrous solid tablet comprising calcium formate, an organic acid different from formic acid, and a basic source of calcium,
wherein the bolus falls and dissolves in ruminal juice of rumen of the ruminant animal so that i) calcium formate is absorbed by the ruminal wall, and ii) the organic acid reacts with the basic source of calcium to form an organic salt of calcium that is absorbed by the ruminal wall.

2. Method of claim 1, wherein the basic source of calcium is calcium carbonate, and the organic acid reacts with calcium carbonate by effervescence to generate carbone dioxide and the organic salt of calcium.

3. Method of claim 2, wherein the organic acid is citric acid, so that citric acid reacts with calcium carbonate by effervescence to generate carbone dioxide and calcium citrate that is absorbed by the ruminal wall.

4. Method of claim 1, wherein the basic source of calcium only reacts partially with the organic acid so that a part of the basic source of calcium is absorbed by the ruminal wall.

5. Method of claim 1, wherein the basic source of calcium is absorbed by the ruminal wall, almost in part.

6. Method of claim 2, wherein calcium carbonate represents from 35% to 40% by weight of the weight of the first bolus, and calcium formate represents from 5% to 15% by weight of the weight of the first bolus.

7. Method of claim 1, comprising a step of administering a second bolus in the form of an anhydrous solid tablet that comprises calcium carbonate and calcium formate, wherein the first bolus releases calcium in the body of the ruminant animal at a greater rate than the second bolus does.

8. Method of claim 7, wherein calcium carbonate represents from 5% to 15% by weight of the weight of the second bolus, and calcium formate represents from 50% to 55% by weight of the weight of the second bolus.

9. Method of claim 7, wherein the first and second boluses are concomitantly administered to the ruminant via oral route.

10. Method of claim 7, wherein the first bolus dissolves in vitro at 40° C. in a ruminal juice in a period of between 5 minutes and 1 hour, and the second bolus dissolves in vitro at 40° C. in the same ruminal juice in a period of between 1 and 10 hours.

11. Method of claim 1 for the treatment or prevention of hypocalcaemia in the ruminant animal in need thereof.

12. Method of claim 1, wherein the ruminant animal is a bovine female at the beginning of lactation.

13. Method of claim 1 for the treatment or prevention of vitular fever in the ruminant animal in need thereof.

14. Method of claim 1, wherein the first bolus and/or the second bolus comprise calcium stearate as a lubricant.

15. Bolus in the form of an anhydrous solid tablet for administration to a ruminant animal, the bolus comprising calcium formate, an organic acid and a basic source of calcium that can react in the presence of water with the organic acid to produce a calcium salt.
